(19) United States
(12) Patent Application Publication
(10) Pub. No.: US 2012/0122948 A1
(43) Pub. Date: May 17, 2012

(54) 3-ALKYL-5-FLUOROINDOLE DERIVATIVES AS MYELOPEROXIDASE INHIBITORS

(75) Inventors: Jalal Soubhye, Bruxelles (BE); François Dufrasne, Bruxelles (BE); Pierre Van Antwerpen, Bruxelles (BE)

(73) Assignee: UNIVERSITE LIBRE DE BEUXELLES, Brussels (BE)

(21) Appl. No.: 13/322,617

(22) PCT Filed: May 27, 2010
(86) PCT No.: PCT/EP2010/057364

§ 371 Date: Jan. 13, 2012

(30) Foreign Application Priority Data
May 27, 2009 (EP) 0916192.9

(51) Int. Cl. A61K 31/4045; C07D 405/06; C12N 9/99; A61P 25/16; A61P 25/00; A61P 25/28; A61P 11/00; C07D 209/14; A61P 9/10

(52) U.S. Cl. 514/414; 548/504; 548/468; 514/415; 435/184

(57) ABSTRACT

The invention relates to a compound of formula (Ia)

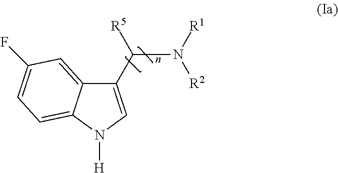

wherein
n is an integer between 2 and 10,
R¹ and R² independently represent a substituent selected from the group consisting of hydrogen, C₁-C₁₀ alkyl, C₃-C₁₀ cycloalkyl and aminoalkyl, or R¹ and R² are taken together with the nitrogen atom to which they are attached to form a four to ten-membered heterocycle,
R⁵ represents independently in each of the n units a substituent selected from the group consisting of hydrogen, C₁-C₁₀ alkyl, halogen, alkoxy, aminoalkyl and alkylamino;
   or a pharmaceutically acceptable salt thereof, with the proviso that the 5-fluorotryptamine is excluded, for the treatment or the prophylaxis of neuroinflammatory diseases or disorders. The invention also relates to a pharmaceutical composition, a method for inhibiting myeloperoxidase enzyme activity, to a method for inhibiting Low density lipoproteins oxidation.

### FIELD OF THE INVENTION

The invention relates to compounds inhibiting the enzyme myeloperoxidase. In particular, the invention refers to 3-alkyl-5-fluoroindole derivatives as myeloperoxidase inhibitors and use thereof in therapy.

### BACKGROUND OF THE INVENTION

Myeloperoxidase (MPO) is a heme-containing enzyme belonging to the peroxidase superfamily which catalyzes the reduction of hydrogen peroxide. Peroxidase enzymes can be found in plants, fungi or animals. Among animal peroxidases, the lactoperoxidase, thyroïde peroxidase, eosinophile peroxidase and myeloperoxidase have been investigated. Myeloperoxidase is present in primary granules of neutrophils and to a lesser extent in monocytes. It catalyzes the synthesis of hypochlorous acid (an oxidative agent) in presence of chloride ions and hydrogen peroxide. The initial role is to favour the elimination of pathogenic agents during phagocytose.

Nevertheless, when acute inflammatory conditions appear, the neutrophils release the so-called “circulating myeloperoxidase”. The involvement of myeloperoxidase in the atherosclerosis process has been studied. Indeed, this enzyme seems to have a major impact at different stages of atherosclerosis and some markers of its activity, such as 3-chlorotyrosine, have been highlighted in atherome plates. Moreover, the myeloperoxidase plasmatic content predicts the appearance of cardiovascular disorders in patients suffering of instable angina pectoris. In atherosclerosis, the “circulating” myeloperoxidase oxidizes apoB100 of LDLs (Low density lipoproteins) and apoAl of HDLs (High density lipoproteins) in physiological conditions.

Klebanoff reviewed the literature related to the potential influence of myeloperoxidase in various pathologies (Klebanoff, S. J., Myeloperoxidase : friend and foe, J. Leuk. Bio., 2005, 77, 598-625). Hence, in carcinogenesis, the enzyme myeloperoxidase is able to chlorinate DNA base pairs, producing for example 5-chloro-2′-deoxycytosine, 5-chlorouracile or 8-chloro-2′-deoxyguanosine. These myeloperoxidase specific markers may be incorporated to DNA and induce mutagenesis (Henderson et al., Molecular chlorine generated by the myeloperoxidase-hydrogen peroxide-chloride system of phagocytes produces 5-chlorocytosine in bacterial RNA, J. Biol. Chem. 1999, 274, 33440-33448; Masuda et al., Chlorination of guanosine and other nucleosides by hypochlorous acid and myeloperoxidase of activated human neutrophils, J. Biol. Chem. 2001, 276, 40486-40496; Takeshita et al., Myeloperoxidase generates 5-chlorouracil in human atherosclerotic tissue, J. Biol. Chem. 2006, 281, 3096-3104).

Another involvement of myeloperoxidase is its oxidative function in glomerulonephritis which has been intensively studied by Stenvinkel et al. (Maruyama et al., Inflammation and oxidative stress in ESRD—the role of myeloperoxidase, J. Nephrol. 2004, 17, S72-S76). In iodiopathic pulmonar fibrosis, Cantin et al. (Oxidant-mediated epithelial cell injury in idiopathic pulmonary fibrosis, J. Clin. Invest. 1987, 79, 1665-1673) have demonstrated the involvement of myeloperoxidase in damages occuring into epithelial cells. Concerning central nervous system, Choi et al. (Ablation of the inflammatory enzyme myeloperoxidase mitigates features of Parkinson's disease in mice, J. Neurosc. 2005, 25, 6594-6600) have highlighted that the enzyme myeloperoxidase plays a major role in Parkinson's disease while Crawford at al. (Association between Alzheimer's disease and a functional polymorphism in the myeloperoxidase gene, Exp. Neurol., 2001, 167, 456-459) reported the same for Alzheimer's disease. Recently, human-MPO mouse model showed a significant accumulation of myeloperoxidase in brain damage in Alzheimer's disease (Maki et al., Aberrant expression of myeloperoxidase in astrocytes promotes phospholipid oxidation and memory deficits in a mouse model of Alzheimer disease, J. Biol. Chem., 2009, 284, 3158-3169).

EP 1 499 613 discloses thioxanthine derivatives as myeloperoxidase inhibitors. These derivatives are useful for the treatment of diseases or disorders in which inhibition of the enzyme myeloperoxidase is beneficial.

Jantschko et al. (Exploitation of the unusual thermodynamic properties of human myeloperoxidase in inhibitor design, Biochemical Pharmacology, 2005, 69, 1149-1157) described indole and tryptamine derivatives, i.a. 5-fluorotryptamine and 5-chlorotryptamine, having the ability to moderately affect the chlorinating activity of MPO (IC₅₀ higher than 0.7 μM). However, in order to achieve a therapeutic use of such compounds, their inhibiting character has to be strongly enhanced and their IC₅₀ value has to be dramatically decreased.

There is a need for new myeloperoxidase inhibitors having enhanced inhibiting character. There is in particular a need for new 5-fluoroindole derivatives that can be suitable for the treatment of diseases or disorders in which inhibition of myeloperoxidase is beneficial.

### SUMMARY OF THE INVENTION

The applicant surprisingly found that new 3-alkyl-5-fluoroindole derivatives show excellent inhibition properties towards myeloperoxidase.

In a first aspect of the invention, compounds or pharmaceutically acceptable salts thereof, for the treatment or the prophylaxis of neuroinflammatory diseases or disorders are provided. Said compounds are of formula (Ia)

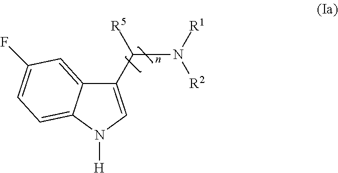

wherein
n is an integer between 2 and 10,
R¹ and R² independently represent a substituent selected from the group consisting of hydrogen, C₁-C₁₀ alkyl, C₃-C₁₀ cycloalkyl, and aminoalkyl, or R¹ and R² are taken together with the nitrogen atom to which they are attached to form a four to ten-membered heterocycle,
R⁵ represents independently in each of the n units a substituent selected from the group consisting of hydrogen, C₁-C₁₀ alkyl, halogen, alkoxy, aminoalkyl, and alkylamino;
   or a pharmaceutically acceptable salt thereof, with the proviso that the 5-fluorotryptamine is excluded. The applicant showed that the 3-alkyl-5-fluoroindole derivatives according to the present invention strongly inhibit myeloperoxidase. The low IC₅₀ values observed against MPO for said compounds of the present invention allow their therapeutic use in diseases in which a decrease of MPO activity is beneficial. Furthermore, the present invention also provides a method for inhibiting myeloperoxidase enzyme activity characterised in that said method comprises the step of adding a compound according to the present invention to a medium containing said enzyme, said compound according to the present invention being added in a concentration effective to inhibit the activity of said enzyme.

In a further aspect of the present invention, a pharmaceutical composition is provided. In particular, said pharmaceutical composition comprises a therapeutically effective amount of a compound according to the present invention, or a pharmaceutically acceptable salt thereof, in admixture with a pharmaceutically acceptable adjuvant, diluent or carrier. Said pharmaceutical composition may be used for the treatment or prophylaxis of neuroinflammatory diseases or disorders.

In another aspect, the present invention provides a method for the treatment of atherosclerosis. In yet another aspect, the present invention provides a method for inhibiting low density lipoproteins oxidation.

### BRIEF DESCRIPTION OF THE DRAWINGS

FIG. 1 represents several chemical synthesis pathways for the preparation of compounds according to the invention.

FIG. 2 represents a graph showing the inhibition capability (expressed as 1/IC₅₀) of various compounds of the invention towards myeloperoxidase enzyme.

FIG. 3 represents a graph showing the inhibition capability (expressed as 1/IC₅₀) in function of chain length n of the compound of formula (Ia).

### DETAILED DESCRIPTION OF THE INVENTION

Whenever the term “substituted” is used in the present invention, it is meant to indicate that one or more hydrogen on the atom indicated in the expression using “substituted” is replaced with a selection from the indicated group, provided that the indicated atom's normal valence is not exceeded, and that the substitution results in a chemically stable compound, i.e. a compound that is sufficiently robust to survive isolation to a useful degree of purity from a reaction mixture, and formulation into a therapeutic agent. Substituents may be selected from but not limited to, for example, the group comprising alkyl, cycloalkyl, aryl, halogen, hydroxyl, nitro, amido, carboxy, amino, cyano. The term “nitro” as used herein refers to the group —NO₂. The term “cyano” as used herein refers to the group —CN. The term “hydroxyl” refers to the group —OH. The term “amido” refers to the group —C(O)—NH—. The term “carboxy” refers to the group —C(O)O—.

The term “alkyl” by itself or as part of another substituent refers to a hydrocarbyl radical of formula CₙH₂ₙ₊₁ wherein n is a number greater than or equal to 1. Generally, alkyl groups of the present invention comprise from 1 to 10 carbon atoms, preferably from 1 to 6 carbon atoms, more preferably from 1 to 4 carbon atoms, still more preferably 1 to 3 carbon atoms. Alkyl groups may be linear or branched and may be substituted. When a subscript is used herein following a carbon atom, the subscript refers to the number of carbon atoms that the named group may contain. Thus, for example, C₁₋₄alkyl means an alkyl of one to four carbon atoms. For example, the “C₁-C₁₀ alkyl” refers but is not limited to methyl, ethyl, n-propyl, i-propyl, n-butyl, i-butyl, s-butyl, t-butyl, 1-pentyl; 2-pentyl, 3-pentyl, i-pentyl, neo-pentyl, t-pentyl, 1-hexyl, 2-hexyl, 3-hexyl, 1-methyl-1-ethyl-n-pentyl, 1,1,2-tri-methyl-n-propyl, 1,2,2-trimethyl-n-propyl, 3,3-dimethyl-n-butyl, 1-heptyl, 2-heptyl, 1-ethyl-1,2-dimethyl-n-propyl, 1-ethyl-2,2-dimethyl-n-propyl, 1-octyl, 3-octyl, 4-methyl-3-n-heptyl, 6-methyl-2-n-heptyl, 2-propyl-1-n-heptyl, 2,4,4-trimethyl-1-n-pentyl, 1-nonyl, 2-nonyl, 2,6-dimethyl-4-n-heptyl, 3-ethyl-2,2-dimethyl-3-n-pentyl, 3,5,5-trimethyl-1-n-hexyl, 1-decyl, 2-decyl, 4-decyl, 3,7-dimethyl-1-n-octyl, 3,7-dimethyl-3-n-octyl. For example, the term “C₁-C₆ alkyl” refers to, but is not limited to, methyl, ethyl, n-propyl, i-propyl, n-butyl, i-butyl, s-butyl, t-butyl, 1-pentyl, 2-pentyl, 3-pentyl, i-pentyl, neo-pentyl, t-pentyl, 1-hexyl, 2-hexyl, 3-hexyl, 1-methyl-1-ethyl-n-pentyl, 1,1,2-tri-methyl-n-propyl, 1,2,2-trimethyl-n-propyl, 3,3-dimethyl-n-butyl. The term “C₁-C₂ alkyl” refers to methyl, ethyl.

The term “four to ten-membered heterocycle” as used herein refers to a four to ten-membered heterocycle comprising at least one nitrogen atom. Preferably, said heterocycle is four to eight-membered heterocycle, more preferably five to six-membered heterocycle. Preferably, said “heterocycle” can be a five or six-membered nitrogen heterocycle. Said “heterocycle” can optionally be substituted by one or more substituent(s) such as alkyl, alkoxy, aryl or halogen. Said “heterocycle” may further comprise another heteroatom such as sulfur, oxygen, phosphorus or nitrogen. Said “heterocycle” can be aromatic. Non-limiting examples of “four to ten-membered heterocycle” may be azetidine, pyrrolidine, piperidine, piperazine, azepane, azocane, methylpiperidine, pyrrole, indole, isoindole, pyridine, triazinane, triazine, azocine, azaphosphinane, morpholine, thiomorpholine, oxazinane, thiazinane, azaphosphinine, thiazine, or oxazine. The term “aryl” as used herein refers to a polyunsaturated, aromatic hydrocarbyl group having a single ring (i.e. phenyl) or multiple aromatic rings fused together (e.g. naphtyl) or linked covalently, typically containing 6 to 10 carbon atoms, wherein at least one ring is aromatic. Non-limiting examples of aryl comprise phenyl, biphenylyl, biphenylenyl, tetralinyl, azulenyl, naphthalenyl, indenyl, acenaphtenyl, phenanthryl, indanyl, pyrenyl. The aryl ring can optionally be substituted by one or more substituent(s).

The term “aminoalkyl” refers to the group —NR^{b}R^{c} wherein R^{b} and R^{c} represent independently hydrogen or alkyl or substituted alkyl as defined herein.

The term “alkoxy” as used herein refers to a radical having the formula —OR^{d} wherein R^{d} is alkyl as described above. For example, alkoxy may be C₁₋₆ alkoxy. Non-limiting examples of suitable alkoxy include methoxy, ethoxy, propoxy, butoxy, n-butoxy, isobutoxy, sec-butoxy, n-pentoxy, isopentoxy, sec-pentoxy, t-pentoxy, or hexyloxy.

The term “halogen” as used herein refers to chloride, fluoride, iodide, or bromide.

The term “cycloalkyl” as used herein is a cyclic alkyl group, that is to say, a monovalent, saturated, or unsaturated hydrocarbyl group having 1 or 2 cyclic structure. Cycloalkyl includes all saturated hydrocarbon groups containing 1 to 2 rings, including monocyclic or bicyclic groups. Cycloalkyl groups may comprise 3 or more carbon atoms in the ring and generally, according to this invention comprise from 3 to 10 carbon atoms. The term “C₃-C₁₀ cycloalkyl” as used herein refers to a cycloalkyl groups comprising from 3 to 10 carbon atoms. For example, the term may include cyclopropane, cyclobutane, cyclopentane, cyclohexane, cycloheptane, cyclooctane, cyclononane, or cyclodecane.

The term “alkylamino” by itself or as part of another substituent refers to a group consisting of an amino group attached to one or two independently selected and optionally substituted alkyl groups, cycloalkyl groups i.e., alkyl amino refers to —N(R^{e})(R^{f}) wherein R^{e} and R^{f} are each independently selected from hydrogen, cycloalkyl, or alkyl. Alkylamino include mono-lower alkyl amino group (e.g. mono-C₁₋₆alkylamino group such as methylamino and ethylamino), di-lower alkylamino group (e.g. di-C₁₋₆alkylamino group such as dimethylamino and diethylamino). Non-limiting examples of suitable alkylamino groups also include n-propylamino, isopropylamino, n-butylamino, isobutylamino, sec-butylamino, tert-butylamino, pentylamino, n-hexylamino, di-n-propylamino, diisopropylamino, ethylmethylamino, methyl-n-propylamino, methyl-i-propylamino, n-butylmethylamino, i-butylmethylamino, t-butylmethylamino, ethyl-n-propylamino, ethyl-i-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “IC₅₀” as used herein refers to the half maximal inhibitory concentration. IC₅₀ represents the concentration of a compound that is required for 50% inhibition of an enzyme in vitro. In the context of the present invention, IC₅₀ values were determined against myeloperoxidase enzyme.

The term “n unit” as used herein refers to the group —(CHR⁵)—. Hence, the term “R⁵ represents independently in each of the n units a substituent” as used herein means that in each “n unit”, i.e. in each —(CHR⁵)— group, R⁵ may be one of the cited substituents, independently of the other or adjacent “n unit” contained in said compound.

In a first aspect, the present invention relates to compounds of formula (Ia)

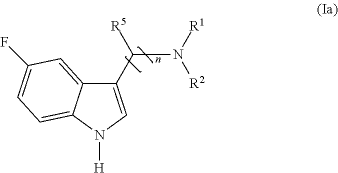

wherein
n is an integer between 2 and 10,
R¹ and R² independently represent a substituent selected from the group consisting of hydrogen, C₁-C₁₀ alkyl, C₃-C₁₀ cycloalkyl and aminoalkyl, or R¹ and R² are taken together with the nitrogen atom to which they are attached to form a four to ten-membered heterocycle,
R⁵ represents independently in each of the n units a substituent selected from the group consisting of hydrogen, C₁-C₁₀ alkyl, halogen, alkoxy, aminoalkyl, and alkylamino;
   or pharmaceutically acceptable salts thereof, with the proviso that 5-fluorotryptamine is excluded, for the treatment or the prophylaxis of diseases or disorders for neuroinflammatory disorders. The term “5-fluorotryptamine” also refers to a compound of formula (Ia) wherein n=2, R¹, R² are hydrogen and R⁵ is hydrogen in each of the n units. The compound 5-fluorotryptamine can also be named 2-(5-fluoro-1H-indol-3-yl)ethyl-1-amine; 3-(2-aminoethyl)-5-fluoro-1H-indole.

The present invention also relates to the use of a compound of formula (Ia)

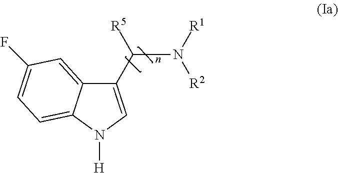

wherein
n is an integer between 2 and 10,
R¹ and R² independently represent a substituent selected from the group consisting of hydrogen, C₁-C₁₀ alkyl, C₃-C₁₀ cycloalkyl and aminoalkyl, or R¹ and R² are taken together with the nitrogen atom to which they are attached to form a four to ten-membered heterocycle,
R⁵ represents independently in each of the n units a substituent selected from the group consisting of hydrogen, C₁-C₁₀ alkyl, halogen, alkoxy, aminoalkyl, and alkylamino;
   or pharmaceutically acceptable salts thereof, with the proviso that 5-fluorotryptamine is excluded, in the manufacture of a medicament for the treatment or prophylaxis of neuroinflammatory diseases or disorders.

n may be an integer between 2 and 6. Hence, n can be 2, 3, 4, 5 or 6, or a value in the range between any two of the aforementioned values. Preferably, n may be an integer between 2 and 4. Alternatively, n may be an integer between 2 and 5.

In a preferred embodiment, R⁵ may represent independently in each of the n units a substituent selected from the group consisting of hydrogen and C₁-C₆ alkyl. In another embodiment, R¹ and R² may independently represent a substituent selected from the group consisting of hydrogen and C₁-C₆ alkyl, or R¹ and R² may be taken together with the nitrogen atom to which they are attached to form a heterocycle selected from the group consisting of azetidine, pyrrolidine, piperidine, piperazine, azepane and azocane. In another embodiment, R⁵ may represent independently in each of the n units a substituent selected from the group consisting of hydrogen and C₁-C₆ alkyl; and R¹ and R² may independently represent a substituent selected from the group consisting of hydrogen and C₁-C₆ alkyl, or R¹ and R² may be taken together with the nitrogen atom to which they are attached to form a heterocycle selected from the group consisting of azetidine, pyrrolidine, piperidine, piperazine, azepane and azocane.

In a preferred embodiment, R⁵ may represent independently in each of the n units a substituent selected from the group consisting of hydrogen and C₁-C₂ alkyl. Preferably, R⁵ may be hydrogen in each of the n units. In another preferred embodiment, R¹ and R² may independently represent a substituent selected from the group consisting of hydrogen and C₁-C₂ alkyl, or R¹ and R² may be taken together with the nitrogen atom to which they are attached to form a heterocycle selected from the group consisting of pyrrolidine and piperazine. R⁵ may represent independently in each of the n units a substituent selected from the group consisting of hydrogen and C₁-C₂ alkyl; and R¹ and R² may independently represent a substituent selected from the group consisting of hydrogen and C₁-C₂ alkyl, or R¹ and R² may be taken together with the nitrogen atom to which they are attached to form a heterocycle selected from the group consisting of pyrrolidine and piperazine. R⁵ may be in each of the n units a hydrogen; and R¹ and R² may independently represent a substituent selected from the group consisting of hydrogen and C₁-C₂ alkyl, or R¹ and R² may be taken together with the nitrogen atom to which they are attached to form a pyrrolidine.

In a preferred embodiment, said compound may be 3-(3-Aminopropyl)-5-fluoro-[1H]-indole of formula (II)

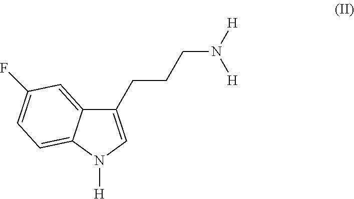

Said compound of formula (II) is equivalent to a compound of general formula (Ia) wherein n is 3, R⁵ is hydrogen in each of the n units, and R¹ and R² are hydrogen.

In a preferred embodiment, said compound may be 3-(4-Aminobutyl)-5-fluoro-[1H]-indole of formula (III)

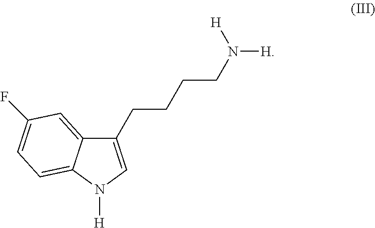

Said compound of formula (III) is equivalent to a compound of general formula (Ia) wherein n is 4, R⁵ is hydrogen in each of the n units, and R¹ and R² are hydrogen.

In a preferred embodiment, said compound may be 5-Fluoro-3-[2-(1-pyrrolidinyl)ethyl]-[1H]-indole of formula (IV)

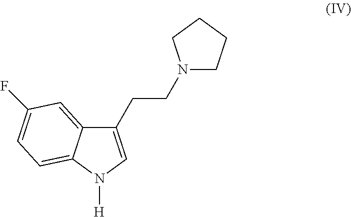

Said compound of formula (IV) is equivalent to a compound of general formula (Ia) wherein n is 2, R⁵ is hydrogen in each of the n units, and R¹ and R² are taken together with the nitrogen atom to which they are attached to form a pyrrolidine.

In a preferred embodiment, said compound may be N,N-Dimethyl-3-(2-aminoethyl)-5-fluoro-1H-indole of formula (V)

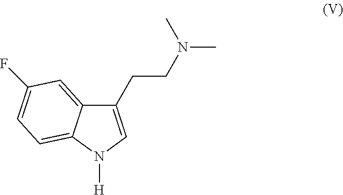

Said compound of formula (V) is equivalent to a compound of general formula (Ia) wherein n is 2, R⁵ is hydrogen in each of the n units, and R¹ and R² are methyl groups.

In a preferred embodiment, said compound may be 3-(5-aminopentyl)-5-fluoro-[1H]-indole oxalate of formula (VI)

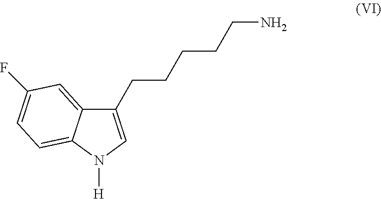

Said compound of formula (VI) is equivalent to a compound of general formula (Ia) wherein n is 5, R⁵ is hydrogen in each of the n units, and R¹ and R² are hydrogen.

Alternatively, the compound may be 3-(6-aminohexyl)-5-fluoro-[1H]-indole oxalate of formula (VII)

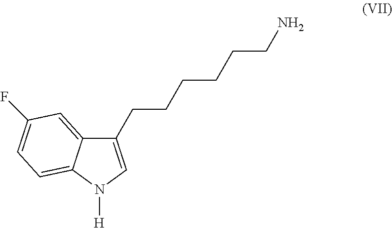

Said compound of formula (VII) is equivalent to a compound of general formula (Ia) wherein n is 6, R⁵ is hydrogen in each of the n units, and R¹ and R² are hydrogen.

In a preferred embodiment, said compound may be selected from the group consisting of (3-(3-Aminopropyl)-5-fluoro-[1H]-indole (3-(4-Aminobutyl)-5-fluoro-[1H]-indole (5-Fluoro-3-[2-(1-pyrrolidinyl)ethyl-[1H]-indole (N,N-Dimethyl-3-(2-aminoethyl)-5-fluoro-[1H]-indole and 3-(5-aminopentyl)-5-fluoro-1H-indole oxalate or a pharmaceutically acceptable salt thereof. In particular, said compound may be selected from the group consisting of (3-(3-Aminopropyl)-5-fluoro-[1H]-indole (3-(4-Aminobutyl)-5-fluoro-[1H]-indole, (5-Fluoro-3-[2-(1-pyrrolidinyl)ethyl]-[1H]-indole and 3-(5-aminopentyl)-5-fluoro-[1H]-indole oxalate or a pharmaceutically acceptable salt thereof. More in particular, said compound may be (3-(4-Aminobutyl)-5-fluoro-[1H]-indole or 3-(5-aminopentyl)-5-fluoro-[1H]-indole oxalate or a pharmaceutically acceptable salt thereof.

In a preferred embodiment, said compound according to the present invention may have an IC₅₀ equal or less than 0.2 μM, preferably less than 0.15 μM, and more preferably less than 0.1 μM against myeloperoxidase enzyme. In particular, said compound according to the present invention may have an IC₅₀ lower than 25 nM against myeloperoxidase enzyme.

The compounds of the present invention may be in the form of salts, in particular acid addition salts. Suitable salts include those formed with both organic and inorganic acids. Such acid addition salts will normally be pharmaceutically acceptable salts although salts of non-pharmaceutically acceptable acids may be of utility in the preparation and purification of the compound of the present invention. Hence, preferred salts include those formed from hydrochloric, hydrobromic, trifluoroacetic, sulphuric, oxalic, phosphoric, citric, tartaric, lactic, pyruvic, acetic, succinic, fumaric, maleic, methanesulphonic, p-toluenesulfonic, formic, adipic, glycolic, aspartic, malic, oleic, nicotinic, saccharinic and benzenesulphonic acids.

The compounds according to the invention represented by the formula (Ia) or pharmaceutically acceptable salts thereof, are indicated for use in the treatment or prophylaxis of diseases or disorders in which modulation of the activity of the enzyme myeloperoxidase is beneficial. In particular, linkage of myeloperoxidase activity to disease has been demonstrated in neuroinflammatory diseases. Therefore, the compound of the present invention is particularly indicated for use in the treatment of neuroinflammatory disorders or diseases in mammals including human. Such diseases or disorders will be readily apparent to the man skilled in the art.

Disorders or diseases that may be specifically mentioned include multiple sclerosis, Alzheimer's disease, Parkinson's disease, amyotrophic lateral sclerosis and stroke, as well as other inflammatory diseases or disorders such as asthma, chronic obstructive pulmonary disease, cystic fibrosis, idiopathic pulmonary fibrosis, acute respiratory distress syndrome, sinusitis, rhinitis, psoriasis, dermatitis, uveitis, gingivitis, atherosclerosis, inflammatory bowel disease, renal glomerular damage, liver fibrosis, sepsis, proctitis, rheumatoid arthritis, and inflammation associated with reperfusion injury, spinal cord injury and tissue damage/scarring/adhesion/rejection. Lung cancer has also been suggested to be associated with high MPO levels. The compounds are also expected to be useful in the treatment of pain.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the diseases or disorders.

In particular, compounds of the present invention are suitable for the use in the treatment of multiple sclerosis, atherosclerosis, Alzheimer's disease, chronic pulmonar disease, chronic inflammatory syndromes linked to joints or Parkinson's disease. Therefore, the present invention relates to the use of a compound or pharmaceutically acceptable salts thereof according to the present invention for use in the manufacture of a medicament for the treatment of multiple sclerosis, atherosclerosis, Alzheimer's disease, chronic pulmonar disease, chronic inflammatory syndromes linked to joints or Parkinson's disease. Hence, compounds of the present invention may be useful for one or more of the above-mentioned diseases or disorders. In particular, said compounds may be suitable for the treatment of cardiovascular diseases such as atherosclerosis. Said compounds may be used for the manufacture of a medicament for use in the treatment of atherosclerosis.

For the above mentioned therapeutic indications, the dosage administered will, of course, vary with the compound employed, the mode of administration and the treatment desired. However, in general, satisfactory results may be obtained when the compounds are administered at a dosage of the solid form of between 0.1 mg and 2000 mg per day.

The present invention also provides a method for inhibiting myeloperoxidase enzyme activity characterised in that said method comprises the step of adding a compound of formula (Ia), with the proviso that 5-fluorotryptamine is excluded, to a medium containing said myeloperoxidase enzyme, said compound of formula (Ia) being added in a concentration effective to inhibit the activity of said enzyme. Said medium containing said myeloperoxidase enzyme may be a phosphate buffer. The pH of said medium may be between 7 and 8, preferably between 7.2 and 7.6. Preferably, the pH of said medium may be approximately 7.4. The concentration of the compound of formula (Ia) effective to inhibit the activity of the enzyme myeloperoxidase may be below 0.2 μM, preferably below 0.15 μM, more preferably below 25 nM. A concentration of a compound is considered as effective to inhibit the activity of an enzyme when such concentration inhibit 50% of the enzyme activity.

In another aspect of the present invention, a pharmaceutical composition is provided. In one embodiment, the pharmaceutical composition comprises a therapeutically effective amount of a compound according to the present invention with the proviso that 5-fluorotryptamine is excluded, or a pharmaceutically acceptable salt thereof, in admixture with a pharmaceutically acceptable adjuvant, diluent or carrier. The term “therapeutically effective amount” refers to dosage that provides the specific pharmacological response for which the drug is administered in a significant number of subjects in need of such treatment. The “therapeutically effective amount” may vary according, for example, the physical condition of the patient, the age of the patient and the severity of the disease.

Said pharmaceutical composition comprises a compound of general formula (la)

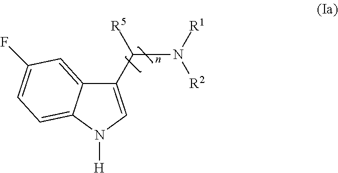

wherein
n is an integer between 2 and 10,
R¹ and R² independently represent a substituent selected from the group consisting of hydrogen, C₁-C₁₀ alkyl, C₃-C₁₀ cycloalkyl and aminoalkyl, or R¹ and R² are taken together with the nitrogen atom to which they are attached to form a four to ten-membered heterocycle,
R⁵ represents independently in each of the n units a substituent selected from the group consisting of hydrogen, C₁-C₁₀ alkyl, halogen, alkoxy, aminoalkyl and alkylamino;
   or a pharmaceutically acceptable salt thereof with the proviso that 5-fluorotryptamine is excluded.

Preferably said pharmaceutical composition may comprise a compound of formula (Ia) wherein n may be an integer between 2 and 6. Hence, n can be 2, 3, 4, 5 or 6, or a value in the range between any two of the aforementioned values. Preferably, n may be an integer between 2 and 4. Alternatively, n may be an integer between 2 and 5.

In another embodiment, said pharmaceutical composition may comprise a compound of formula (Ia) wherein R⁵ may represent independently in each of the n units a substituent selected from the group consisting of hydrogen and C₁-C₆ alkyl. In another embodiment, said pharmaceutical composition may comprise a compound of formula (Ia) wherein R¹ and R² may independently represent a substituent selected from the group consisting of hydrogen and C₁-C₆ alkyl, or R¹ and R² may be taken together with the nitrogen atom to which they are attached to form a heterocycle selected from the group consisting of azetidine, pyrrolidine, piperidine, piperazine, azepane and azocane. Said pharmaceutical composition may comprise a compound of formula (Ia) wherein R⁵ may represent independently in each of the n units a substituent selected from the group consisting of hydrogen and C₁-C₆ alkyl; and R¹ and R² may independently represent a substituent selected from the group consisting of hydrogen and Cₗ-C₆ alkyl, or R¹ and R² are taken together with the nitrogen atom to which they are attached to form a heterocycle selected from the group consisting of azetidine, pyrrolidine, piperidine, piperazine, azepane and azocane.

In a preferred embodiment, said pharmaceutical composition may comprise a compound of formula (Ia) wherein R⁵ may represent independently in each of the n units a substituent selected from the group consisting of hydrogen and C₁-C₂ alkyl. Preferably, R⁵ may be in each of the n units a hydrogen. In another preferred embodiment, said pharmaceutical composition may comprise a compound of formula (Ia) wherein R¹ and R² may independently represent a substituent selected from the group consisting of hydrogen and C₁-C₂ alkyl, or R¹ and R² may be taken together with the nitrogen atom to which they are attached to form a heterocycle selected from the group consisting of pyrrolidine and piperazine. Said pharmaceutical composition may comprise a compound of formula (Ia) wherein R⁵ may represent independently in each of the n units a substituent selected from the group consisting of hydrogen and C₁-C₂ alkyl; and R¹ and R² may independently represent a substituent selected from the group consisting of hydrogen, C₁-C₂ alkyl, or R¹ and R² may be taken together with the nitrogen atom to which they are attached to form a heterocycle selected from the group consisting of pyrrolidine and piperazine. Said pharmaceutical composition may comprise a compound of formula (Ia) wherein R⁵ may be in each of the n units a hydrogen; and R¹ and R² may independently represent a substituent selected from the group consisting of hydrogen, C₁-C₂ alkyl, or R¹ and R² may be taken together with the nitrogen atom to which they are attached to form a pyrrolidine.

In a preferred embodiment, said pharmaceutical composition may comprise a compound of formula (Ia) wherein said compound may be selected from the group consisting of (3-(3-Aminopropyl)-5-fluoro-[1H]-indole, (3-(4-Aminobutyl)-5-fluoro-[1H]-indole, (5-Fluoro-3-[2-(1-pyrrolidinyl)ethyl]-[1H-indole, (N,N-Dimethyl-3-(2-aminoethyl)-5-fluoro-[1H-indole, and 3-(5-aminopentyl)-5-fluoro-1H-indole oxalate or a pharmaceutically acceptable salt thereof as defined above. In particular, said pharmaceutical composition may comprise a compound of formula (Ia) selected from the group consisting of (3-(3-Aminopropyl)-5-fluoro-[1H]-indole, 3-(4-Aminobutyl)-5-fluoro-[1H]-indole, (5-Fluoro-3-[2-(1-pyrrolidinyl)ethyl]-[1H]-indole and 3-(5-aminopentyl)-5-fluoro-1H-indole oxalate or a pharmaceutically acceptable salt thereof. More in particular, said pharmaceutical composition may comprise a compound of formula (Ia) selected from the group consisting of 3-(4-Aminobutyl)-5-fluoro-[1H]-indole and 3-(5-aminopentyl)-5-fluoro-1H-indole oxalate or a pharmaceutically acceptable salt thereof.

In a preferred embodiment, said pharmaceutical composition may comprise a compound of formula (Ia) having an IC₅₀ equal or less than 0.2 μM, preferably less than 0.15 μM, and more preferably less than 0.1 μm against myeloperoxidase enzyme. In particular, said pharmaceutical composition may comprise a compound of formula (Ia) having an IC₅₀ lower than 25 nM against myeloperoxidase enzyme.

Said compounds represented by the formula (Ia), and pharmaceutically acceptable salts thereof, are useful since they possess a low IC₅₀ value. Therefore, the compounds of formula (II), (III), (IV), (V), or (VI) are useful since they possess pharmacological activity in diseases where a decrease of MPO activity is beneficial.

The pharmaceutical composition may comprise less than 80% and more preferably less than 50% of a compound of formula (Ia), or a pharmaceutically acceptable salt thereof. Administration of such pharmaceutical composition may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, “Pharmaceuticals—The science of dosage form designs”, M. E. Aulton, Churchill Livingstone, 1988.

Said pharmaceutical composition may be suitable for the treatment or the prophylaxis of neuroinflammatory diseases or disorders as defined above. Preferably, said neuroinflammatory diseases or disorders may be selected from the group consisting of multiple sclerosis, atherosclerosis, Alzheimer's disease, chronic pulmonar disease, chronic inflammatory syndromes linked to joints and Parkinson's disease. In particular, said pharmaceutical composition may be used for the treatment of atherosclerosis.

In another embodiment, the present invention relates to the use of said pharmaceutical composition for the manufacture of a medicament for use in the treatment or the prophylaxis of neuroinflammatory diseases or disorders. The neuroinflammatory diseases or disorders are defined above. In particular, said pharmaceutical composition may be used for the manufacture of a medicament for use in the treatment of atherosclerosis.

The present invention also provides a method for the treatment of atherosclerosis characterised in that said method comprises the step of administering an therapeutically effective amount of a compound according to the present invention or a therapeutcially effective amount of a pharmaceutical composition according to the present invention to a patient in need thereof.

Compounds of the present invention are also suitable for inhibiting Low density lipoproteins (LDL) oxidation. This is demonstrated in the examples. The present invention also provides a method for inhibiting low density lipoproteins (LDL) oxidation characterised in that said method comprises the step of contacting a compound according to the present invention in a medium containing said low density lipoproteins and myeloperoxidase enzyme. Said compound may be added in a concentration effective to inhibit the oxidation of said low density lipoproteins. The concentration effective to inhibit the oxidation of said low density lipoproteins may be lower than 2000 nM. Preferably said concentration may be equal or lower than 1000 nM. Said medium may be a phosphate buffer. The pH of said buffer may be between 7 and 8, preferably between 7.0 and 7.5.

### EXAMPLES

The invention is illustrated, but in no way limited, by the following examples.

Experimental Procedure

¹H- and ¹³C-NMR spectra were taken on a Bruker Avance 300 spectrometer at 293K (frequencies: 300 MHz for ¹H and 75 MHz for ¹³C). δ are given in ppm relative to TMS and the coupling constants are expressed in Hz. IR analyses were performed with a Shimadzu IR-470 spectrophotometer and the peaks data are given in cm⁻¹. All reactions were followed by TLC carried out on Fluka PET-foils silica gel 60 Å with a fluorescence indicator (254 nm) and compounds were visualized by UV and by spraying Van Urk reagent (a 1%_{w/w} para-dimethylaminobenzaldehyde solution in a mixture of concentrated HCl and ethanol (1:1)). Column chromatography was performed with EchoChrom MP silica 63-200, 60 A°. Solutions were dried over Na₂SO₄ and concentrated with a Buchi rotary evaporator and Edwards RV3 vacuum pump at low pressure.

Compounds Synthesis

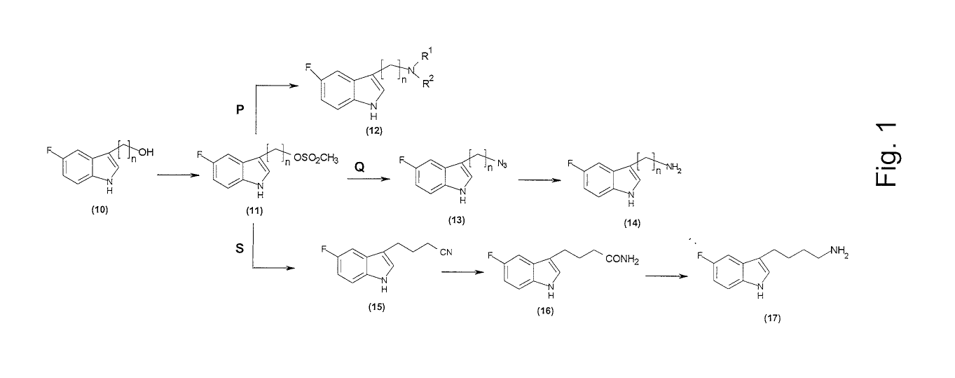

The 3-alkyl-5-fluoroindole derivatives of formula (Ia) according to the present invention can be prepared from 3-(hydroxyalkyl)-5-fluoroindole derivatives (10) as depicted in FIG. 1. The compound (10) was reacted with methane sulphonyl chloride to afford the corresponding methanesulfonate compounds (11) according procedure known in the art. Then, three different chemical synthesis pathways summarized in FIG. 1 can be used. The pathway P corresponds to the reaction of the compound (11) with an amine in dioxane at 100° C. to afford the compound (12). This pathway P was used for the preparation of compounds D, E, F, G, H, J and L (examples 3-8 and 10). The pathway Q mentions the reaction of the compound (11) with NaN₃ in dimethylsulfoxide to afford the corresponding azido compound (13) which then reacted with palladium on charcoal to provide the corresponding primary amine (14). This pathway Q was used in example 1. The pathway S corresponds to the reaction of the compound (11) with NaCN in a mixture of water and dimethylacetamide to afford the corresponding nitrile compound (15) which then reacts with KOH and tBu-OH to provide the corresponding amide compound (16). This latter amide compound (16) then reacts with LiAlH₄ to afford the desired amine (17). The pathway S was used in example 2 and example 9.

### Example 1

### Compound B—Formula (II)

3-(3-Aminopropyl)-5-fluoro-[1H]-indole, compound of formula (II) or of formula (Ia) wherein n=3, Wand R² are hydrogen and R⁵ is hydrogen in each of the n units. Pd on charcoal 10% (50 mg) was added to a solution of 1-azido-3-(5-Fluoroindol-3-yl)propane-1 (1 g, 4.6mmol) in ethanol (20 mL). The suspension was stirred under H₂ (60 psi) overnight. After filtration on celite, the solvent was evaporated. The residue was dissolved in ether, extracted with HCl 0.1 N and the resulting solution was washed with ether. A solution of NaOH 1 N was added (pH=10) and the mixture was extracted with diethylether. The organic layer was washed with water, dried over Na₂SO₄ and evaporated to afford the pure product. (White solids, 0.66 g, 80% yield).

¹H NMR (CDCl₃) δ 8.94 (br s, 1H), 7.28 (m, 2H), 7.01 (d, J=1.2 Hz, 1H), 6.96 (dt, 1H, J=9.0, 2.4 Hz), 2.81 (m, 4H), 1.89 (m, 2H), 1.49 (s, 2H). ¹³C NMR (CDCl₃) δ 157.9 (d, J=232 Hz), 133.1, 127.8 (d, J=10 Hz), 123.3, 116.1 (d, J=5 Hz), 111.9 (d, J=10 Hz), 110.2 (d, J=25 Hz), 103.7 (d, J=24 Hz), 42.1, 33.96, 22.56; IR(KBr) 3762, 3648, 3572, 2660, 2478, 1634, 608 cm⁻¹; HRMS (ESI) calculated for C₁₁H₁₄FN₂ (M+H): 193.1136, found: 193.11365, error: 0.26 ppm.

### Example 2

### Compound C—Formula (III)

3-(4-Aminobutyl)-5-fluoro-[1H]-indole oxalate, oxalic salt of compound of formula (III) or of formula (Ia) wherein n=4, R¹, R² are hydrogen and R⁵ is hydrogen in each of the n units. 4-(5-Fluoro-1H-indol-3-yl)butan-1-amide (7.7 mmol) was dissolved in dioxane (50 mL) and LiAlH₄ (1.0 M solution in dioxane 25 mL) was added. The suspension was refluxed for 12 h and the reaction was quenched with ice and KOH 15% (10 ml). The mixture was filtered through celite and extracted with EtOAc. The organic layer was extracted with HCl 0.1 M and, after decantation, the aqueous phase was washed by EtOAc. KOH 1 M was added to the acid layer (pH=10) and this was extracted with diethylether. The solvent was dried on Na₂SO₄ and evaporated. The residue was dissolved in diethylether and a saturated solution of anhydrous oxalic acid in diethylether was added dropwise. The precipitate was filtered, washed with ether and dried at 40° C. to give a white solid (0.6 g, 26% yield). ¹H NMR (DMSO-d₆) δ 10.96 (br s, 1H), 7.31 (dd, 1H, J=8.7, 4.5 Hz), 7.23 (dd, 1H, J=9.7, 2.7 Hz), 7.18 (s, 1H), 6.86 (dt, 1H, J=7.1, 0.7 Hz), 2.74 (t, 2H, J=6.7, 1.3 Hz), 2.62 (t, 2H, J=6.7, 1.3 Hz), 1.59 (m, 4 H); ¹³C NMR (DMSO-d₆) δ 166.6, 157.9 (d, J=232 Hz), 132.6, 126.9 (d, J=10 Hz), 124.2, 114.1 (d, J=5 Hz), 111.9 (d, J=10 Hz), 108.5 (d, J=25 Hz), 102.7 (d, J=23 Hz), 39.3, 27.5, 26.2, 23.8; IR(KBr) 3686, 3572, 2432, 2356, 2166, 1862, 1634, 1558, 1064, 800 cm⁻¹; HRMS (ESI) calculated for C₁₂H₁₆FN₂ (M+H): 207.1292, found: 207.1292, error: 0.00 ppm

### Example 3

### Compound D—Formula (V)

N,N-Dimethyl-3-(2-aminoethyl)-5-fluoro-[1H]-indole, compound of formula (V) or of formula (Ia) wherein n=2, R⁵ is hydrogen in each of the n units, R¹ and R² are methyl group. A solution of 3-(5-Fluoroindol-3-yl)ethanol methanesulfonate (1 g, 3.6 mmol) in dioxane (5 mL) was added very slowly through an addition funnel to a refluxing solution of dimethylamine (0.26 mol) in dioxane (15 mL) at 100 ° C. After the addition was completed, the reaction medium was stirred at this temperature for 4 h. After cooling, the mixture was treated with water 20 mL and extracted with EtOAc. The organic layer was dried over Na₂SO₄ and evaporated to dryness to afford a crude product. The residue was dissolved in HCl 0.1 N. This solution was washed with diethylether and rendered alkaline (pH=10) with a solution of NaOH 1 N. The mixture was extracted by ether. The organic layer was washed with water, dried over Na₂SO₄ and evaporated to afford white crystals (400 mg, 50% yield)

¹H NMR (CDCl₃) δ8.40 (br s, 1H), 7.19 (m, 2H), 6.99 (s, 1H), 6.88 (dt, 1H, J=7.2, 0.8 Hz), 2.88 (t, 2H, J=7.5 Hz), 2.61 (t, 2H, J=8.7 Hz), 2.33 (s, 6H); ¹³C NMR (CDCl₃) δ 157.9 (d, J=232 Hz), 132.5, 127.5 (d, J=10 Hz), 123.1, 114.1 (d, J=5 Hz), 111.4 (d, J=10 Hz), 110.1 (d, J=25 Hz), 103.2 (d, J=24 Hz), 59.8, 45.1, 23.3; IR(KBr) 3762, 3648, 3572, 2660, 2432, 2356, 2128, 1938, 1520, 1064, 874, 800 cm⁻¹; HRMS (ESI) calculated for C₁₂H₁₆FN₂ (M+H): 207.1292, found: 207.1307, error: 7.24 ppm.

### Example 4

### Compound E—Formula (IV)

5-Fluoro-3-[2-(1-pyrrolidinyl)ethyl]-8 1H]-indole, compound of formula (IV) or of formula (Ia) wherein n=2, R⁵ is hydrogen in each of the n units, and R¹ and R² are taken together with the nitrogen atom to which they are attached to form a pyrrolidine. A solution of 3-(5-Fluoroindol-3-yl)ethanol methanesulfonate (1 g, 3.6 mmol) in dioxane (5 mL) was added very slowly through an addition funnel to a refluxing solution of pyrrolidine (21.8 mL, 0.26 mol) in dioxane (15 mL) at 100° C. After the addition was completed, the reaction medium was stirred at this temperature for 4 h. After cooling, the mixture was treated with water 20 mL and extracted with EtOAc. The organic layer was dried over Na₂SO₄ and evaporated to dryness to afford a crude product. The residue was dissolved in HCl 0.1 N. This solution was washed with diethylether and rendered alkaline (pH=10) with a solution of NaOH 1 N. The mixture was extracted by ether. The organic layer was washed with water, dried over Na₂SO₄ and evaporated to afford brown crystals

¹H NMR (CDCl₃) δ 8.41 (br s, 1H), 7.28 (dd, 1H, J=9.7, 2.7 Hz),7.24 (dd, 1H, J=8.7, 4.5 Hz), 7.06 (d, J=1.2 Hz, 1H), 6.92 (dt, 1H, J=9.3, 2.4 Hz), 2.99 (t, 2H, J=7.2 Hz), 2.84 (t, 2H, J=7.2 Hz), 2.67 (m, 4H), 1.87 (s, 4H); ¹³C NMR (CDCl₃) δ 157.9 (d, J=232 Hz), 133.0, 127.9 (d, J=10 Hz), 123.5, 114.8 (d, J=5 Hz), 111.8 (d, J=10 Hz), 110.4 (d, J=25 Hz), 103.8 (d, J=24 Hz), 57.15, 54.4, 25.2, 23.6; IR (KBr) 3762, 3648, 3572, 2660, 2432, 2356, 2128, 1938, 1520, 1064, 874, 800 cm⁻¹; HRMS (ESI) calculated for C₁₄H₁₈FN₂ (M+H): 233.1449, found: 233.1452, error: 1.33 ppm.

### Example 5

### Compound F

N,N-Diethyl-3-(2-aminoethyl)-5-fluoro-[1H]-indole , compound of formula (Ia) wherein n=2, R⁵ is hydrogen in each of the n units, R¹ and R² are ethyl group. A solution of 3-(5-Fluoroindol-3-yl)ethanol methanesulfonate (1 g, 3.6 mmol) in dioxane (5 mL) was added very slowly through an addition funnel to a refluxing solution of diethylamine (0.26 mol) in dioxane (15 mL) at 100° C. After the addition was completed, the reaction medium was stirred at this temperature for 4h. After cooling, the mixture was treated with water 20 mL and extracted with EtOAc. The organic layer was dried over Na₂SO₄ and evaporated to dryness to afford a crude product. The residue was dissolved in HCl 0.1 N. This solution was washed with diethylether and rendered alkaline (pH≈10) with a solution of NaOH 1 N. The mixture was extracted by ether. The organic layer was washed with water, dried over Na₂SO₄ and evaporated to afford the pure product (white crystals, 455 mg, 50% yield).

¹H NMR (CDCl₃) δ 8.08 (br s, 1H), 7.21 (m, 2H), 7.01 (d, J=1.2 Hz, 1H), 6.88 (dt, 1H, J=7.2, 0.8 Hz), 2.81 (m, 2H), 2.75(m, 2H), 2.62(m, 4H), 1.05(t, 6H, J=7.4 Hz) ; ¹³C NMR (CDCl₃) δ 157.9 (d, J=232 Hz), 132.5, 127.7 (d, J=10 Hz), 123.0, 114.7 (d, J=5 Hz), 111.4 (d, J=10 Hz), 10.9.9 (d, J=25 Hz), 103.5 (d, J=24 Hz), 46.7, 22.6, 11.6; IR(KBr) 3762, 3648, 3572, 2660, 2432, 2356, 2128, 1938, 1520, 1064, 874, 800 cm⁻¹; HRMS (ESI) calculated for C₁₄H₁₉FN₂ (M⁺): 234.1527, found: 234.1528, error: 0.34 ppm.

### Example 6

### Compound G

N-Methyl-3-(3-aminopropyl)-5-fluoro-[1H]-indole tartrate , compound of formula (Ia) wherein n=3, R⁵ and R² are hydrogen, R¹ is methyl group. A solution of 3-(5-Fluoroindol-3-yl)propanol methanesulfonate (1 g, 3.6 mmol) in dioxane (5 mL) was added very slowly through an addition funnel to a refluxing solution of methylamine (0.26 mol) in dioxane (15 mL) at 100° C. After the addition was completed, the reaction medium was stirred at this temperature for 4 h. After cooling, the mixture was treated with water 20 mL and extracted with EtOAc. The organic layer was dried over Na₂SO₄ and evaporated to dryness to afford a crude product. The residue was dissolved in HCl 0.1 N. This solution was washed with diethylether and rendered alkaline (pH=10) with a solution of NaOH 1N. The mixture was extracted by ether. The organic layer was washed with water, dried over Na₂SO₄ and evaporated. The compound has been isolated as a tartrate salt using the following method. After the ether has been evaporated, the residue was heated with a solution of D-tartaric acid (253 mg, 1.4 mmol tartaric acid for 291 mg, 1.4 mmol of compound) in 2-propanol (10 ml). The mixture was cooled and the solid was filtered and dried to afford yellow crystals (545 mg, 40% yield). ¹H NMR (CDCl₃) δ 8.43 (br s, 1H), 7.29 (m, 2H), 7.04 (d, J=1.2 Hz, 1H), 6.96 (dt, 1H, J=9.4, 2.4 Hz), 2.80 (t, 2H, J=7.5 Hz), 2.72 (t, 2H, J=7.2 Hz), 2.50 (s, 3H), 1.94 (m, 2 H), 1.40 (br s, 1H); ¹³C NMR (0001₃) δ 157.9 (d, J=232 Hz), 133.0, 128.0 (d, J=10 Hz), 123.2, 116.5 (d, J=5 Hz), 111.8 (d, J=10 Hz), 110.4 (d, J=25 Hz), 103.8 (d, J=24 Hz), 52.0 (s), 36.6 (s), 30.3 (s), 22.9 (s); IR(KBr) 3762, 3610, 3458, 2660, 2470, 1976, 1558, 836, 646 cm⁻¹; HRMS (ESI) calculated for C₁₂H₁₇FN₂ (M+H): 207.1292, found: 207.13, error: 3.86 ppm.

### Example 7

### Compound H

N,N-Dimethyl-3-(3-aminopropyl)-5-fluoro-[1H]indole tartrate, compound of formula (Ia) wherein n=3, R⁵ is hydrogen in each of the n units; R¹ and R² are methyl group. A solution of 3-(5-Fluoroindol-3-yl)propanol methanesulfonate (1 g, 3.6 mmol) in dioxane (5 mL) was added very slowly through an addition funnel to a refluxing solution of dimethylamine (0.26 mol) in dioxane (15 mL) at 100° C. After the addition was completed, the reaction medium was stirred at this temperature for 4 h. After cooling, the mixture was treated with water 20 mL and extracted with EtOAc. The organic layer was dried over Na₂SO₄ and evaporated to dryness to afford a crude product. The residue was dissolved in HCl 0.1 N. This solution was washed with diethylether and rendered alkaline (pH=10) with a solution of NaOH 1 N. The mixture was extracted by ether. The organic layer was washed with water, dried over Na₂SO₄ and evaporated. The compound has been isolated as a tartrate salt using the following method. After the ether has been evaporated, the residue was heated with a solution of tartaric acid in 2-propanol. The mixture was cooled and the solid was filtered and dried to afford (755 mg, 55% yielded) of white crystals.

¹H NMR (DMSO-d₆) δ 10.92 (br s, 1H), 7.32 (dd, 1H, J=8.7, 4.5 Hz), 7.26 (dd, 1H, J=9.4, 2.4 Hz), 7.22 (d, 1H, J=2.1 Hz), 6.88 (dt, 1H, J=9.2, 2.4 Hz), 3.94 (s, 1H), 2.66 (t, 4H, J=6.9, 8.4 Hz), 2.44 (s, 6H), 1.86 (m, 2H); ¹³C NMR (DMSO-d₆) δ 174.4 (s), 157.9 (d, J=232 Hz), 132.5 (s), 126.8 (d, J=10 Hz,), 124.2, 113.5 (d, J=5 Hz), 111.8 (d, J=10 Hz), 108.5 (d, J=25 Hz), 102.5 (d, J=24 Hz), 71.4 (s), 57.1 (s), 43.1 (s), 25.6 (s), 21.6 (s); IR(KBr) 3762, 3648, 3572, 2660, 2432, 2356, 2128, 1938, 1520, 1064, 874, 800 cm⁻¹; HRMS (ESI) calculated for C₁₃H₁₈FN₂ (M+H): 221.1449, found: 221.14549, error: 2.67 ppm.

### Example 8

### Compound J

N-Propyl-3-(3-aminopropyl)-5-fluoro-[1H]-indole, compound of formula (Ia) wherein n=3, R⁵ and R² are hydrogen, R¹ is propyl group. A solution of 3-(5-Fluoroindol-3-yl)propanol methanesulfonate (1 g, 3.6 mmol) in dioxane (5 mL) was added very slowly through an addition funnel to a refluxing solution of propylamine (0.26 mol) in dioxane (15 mL) at 100° C. After the addition was completed, the reaction medium was stirred at this temperature for 4 h. After cooling, the mixture was treated with water 20 mL and extracted with EtOAc. The organic layer was dried over Na₂SO₄ and evaporated to dryness to afford a crude product. The residue was dissolved in HCl 0.1 N. This solution was washed with diethylether and rendered alkaline (pH=10) with a solution of NaOH 1N. The mixture was extracted by ether. The organic layer was washed with water, dried over Na₂SO₄ and evaporated to afford brown solid (490 mg, 47% yield).

¹H NMR (DMSO-d₆) δ 10.84 (br s, 1H), 7.32 (m, 1H), 7.26 (dd, 1H, J=10.2, 2.4 Hz), 7.16 (s), 6.87 (dt, 1H, J=9.0, 2.1 Hz), 2.66 (t, 2H, J=7.5 Hz), 2.53 (m, 4H, H-1′), 1.73 (m, 2H), 1.39 (m, 2 H), 0.85 (t, 3H, J=7.2 Hz); ¹³C NMR (DMSO-d₆) δ 156.4 (d, J=229 Hz), 135.8 (s), 127.3 (d, J=10 Hz), 124.2 (s), 114.8 (d, J=5 Hz), 112.0 (d, J=10 Hz), 108.7 (d, J=26 Hz), 102.9 (d, J=23 Hz), 51.4 (s), 49.1 (s), 30.2 (s), 22.7 (s), 22.3(C-3), 11.8 (s); IR(KBr) 3400, 3140, 2940, 1550, 1520, 1490, 1420, 1360, 1230, 1190, 1110, 940, 800 cm⁻¹; HRMS (ESI) calculated for C₁₁₄H₁₉FN₂ (M⁺): 234.1527, found: 2234.153, error: 1.28 ppm.

### Example 9

### Compound K

3-(5-Aminopentyl)-5-fluoro-[1H]-indole oxalate, compound of formula (Ia) wherein n=5, R¹ and R² are hydrogen and R⁵ is hydrogen in each of the n units. 4-(5-fluoro-1H-indol-3-yl)pentanamide (1.4 g, 7.6 mmol) was dissolved in dioxan (50 mL) and LiAlH₄ (1.0 M solution in dioxan, 25 mL) was added. The suspension was refluxed for 3 h and the reaction was quenched with ice and (10 mL) KOH 15%. The mixture was filtered through celite and extracted with EtOAc (30 mL). The organic layer was extracted with HCl 0.1 M and, after decantation, the aqueous phase was washed by diethylether. KOH 1 M was added to the acidic layer (pH≈10) and this was extracted with diethylether. The solvent was dried on Na₂SO₄ and evaporated. The residue was dissolved in diethylether and a saturated solution of anhydrous oxalic acid in diethylether was added dropwise. The precipitate was filtered, washed with ether and dried at 40° C. under reduced pressure to give a white solid (0.7 g, 26% yield). ¹H NMR (DMSO-d₆) δ 10.94 (br s), 7.31 (dd, 1H, J=8.7, 4.5 Hz), 7.24 (dd, 1H, J=9.7, 2.7 Hz), 7.18 (s, 1H), 6.86 (dt, 1H, J=7.1, 0.7 Hz), 2.76 (t, 2H, J=6.7, 1.3 Hz), 2.63 (t, 2H, J=6.7, 1.3 Hz), 1.59 (m, 4 H), 1.35 (m); ¹³0 NMR (DMSO-d₆) δ 165.0 (COO), 157.9 (d, J=232 Hz), 132.6 (s), 127.2 (d, J=10 Hz), 124.4 (s), 114.6 (d, J=5 Hz), 111.9 (d, J=10 Hz), 109.0 (d, J=25 Hz), 102.8 (d, J=23 Hz), 38.8, 29.3, 26.9, 25.4, 24.3; IR(KBr) 3686, 3572, 2432, 2356, 2166, 1862, 1634, 1558, 1064, 800 cm⁻¹; HRMS (ESI) calculated for C₁₃H₁₈FN₂ (M+H): 221.1449, found: 221.1470.

### Example 10

### Compound L

3-(6-Aminohexyl)-5-fluoro-[1H]-indole oxalate, compound of formula (Ia) wherein n=6, R¹ and R² are hydrogen and R⁵ is an hydrogen in each of the n units. Palladium on charcoal 10% (50 mg) was added to a solution of 3-(6-azidohexyl)-5-fluoro-1H-indole (1 g, 4.9 mmol) in ethanol (20 mL). The suspension was stirred under H₂ (60 psi) overnight in a Parr hydrogenation apparatus. After filtration on celite, the solvent was evaporated under reduced pressure. The product was dissolved in ether, washed with water, and dried over Na₂SO₄. To this solution, a saturated solution of anhydrous oxalic acid in ether was added and the resulting solid was filtered, washed by ether, and dried to afford yellowish crystals (560 mg, 20% yield). ¹H NMR (DMSO-d₆) δ 10.94 (br s, 1H), 7.31 (dd, 1H, J=8.7, 4.5 Hz), 7.22 (dd, 1H, J=9.7, 2.7 Hz), 7.18 (s, 1H), 6.88 (dt, 1H, J=7.1, 0.7 Hz), 2.69 (t, 2H, J=6.7, 1.3 Hz), 2.62 (t, 2H, J=6.7, 1.3 Hz), 1.60 (m, 4 H), 1.35 (m, 4H); ¹³C NMR (DMSO-d₆) δ 165.0 (COO), 157.9 (d, J=232 Hz), 132.9, 127.3 (d, J=10 Hz), 124.4, 114.6 (d, J=5 Hz), 111.9 (d, J=10 Hz), 109.0 (d, J=25 Hz), 102.8 (d, J=23 Hz), 38.8, 29.3, 26.9, 25.4, 24.3, 22.6; IR(KBr) 3686, 3572, 2432, 2356, 2166, 1862, 1634, 1558, 1064, 800 cm⁻¹; HRMS (ESI) calculated for C₁₄H₂₀FN₂ (M+H): 235.1605, found: 235.1622.

Myeloperoxidase Inhibition (MPO) Assay Procedure

The assay was based on the production of taurine chloramine produced by the MPO/H₂O₂/Cl⁻ system in the presence of a selected inhibitor at defined concentration. The procedure for detecting the inhibition of myeloperoxidase has been described in Van Antwerpen et al. (Development and validation of a screening procedure for the assessment of inhibition using a recombinant enzyme, Talanta, 2008, 75(2), 503-510) and is incorporated herewith by reference. Briefly, the reaction mixture contained the following reagents in a final volume of 200 μl: pH 7.4 phosphate buffer (PO₄³⁻10 mM/NaCl 300 mM), taurine (15 mM), a compound to be tested (up to 20 μM), and the fixed amount of the recombinant MPO (6.6 μl of MPO batch solution diluted 2.5 times, 40 nM). When necessary, the volume was adjusted with water. This mixture was incubated at 37° C. and the reaction initiated with 10.0 μl of H₂O₂ (100 μM). After 5 minutes, the reaction was stopped by the addition of 10 μl of catalase (8 U/μl). To determine the amount of taurine chloramines produced, 50 μl of 1.35 mM solution of thionitrobenzoic acid were added and the volume adjusted to 300 μl with water. Then, the absorbance of the solutions was measured at 412 nm with a microplate reader and the curve of the absorbance as a function of the inhibitor concentration was plotted. IC₅₀ values against myeloperoxidase enzyme were then determined using standard procedures taking into account the absence of hydrogen peroxide as 100% of inhibition and the absence of inhibitors as 0% of inhibition.

5-fluorotryptamine, a compound known in the state of the art, has been tested following the above-mentioned procedure, and an IC₅₀ of 0.2 μM has been obtained. When tested in the above assay, it is particularly remarkable that compounds of the present invention gave IC₅₀ values of less than 0.2 μM.

Table 1 reports the IC₅₀ values obtained with various compounds according to the present invention.

TABLE 1

| Compound | IC₅₀ (μM) | 1/IC₅₀ (μM⁻¹) |
|---|---|---|
| B | 0.050 ± 0.008 | 20 ± 4 |
| C | 0.015 ± 0.004 | 72 ± 19 |
| D | 0.09 ± 0.06 | 14 ± 7 |
| E | 0.04 ± 0.03 | 32 ± 19 |
| F | 0.16 ± 0.08 | 7 ± 3 |
| G | 0.2 ± 0.2 | 6 ± 2 |
| H | 0.13 ± 0.09 | 11 ± 8 |
| J | 0.17 ± 0.03 | 6 ± 1 |
| K | 0.008 ± 0.001 | 122 ± 15 |
| L | 0.26 ± 0.01 | 3.8 ± 0.2 |

The results show very low IC₅₀ values that are decreased compared to the prior art. Indeed, IC₅₀ values lower than 0.025 μM was obtained for compounds C (0.015 μM) and K (0.008 μM). Nanomolar levels was achieved with these compounds, and a decrease of thirteen orders of magnitude was obtained compared to 5-fluorotryptamine. Hence, the compounds of the present invention inhibit the enzyme myeloperoxidase in such a way that has never been expected and reported in the art. The compounds according to the present invention are expected to show powerful therapeutic activity, mainly in diseases or disorders in which an increase of myeloperoxidase content is harmful.

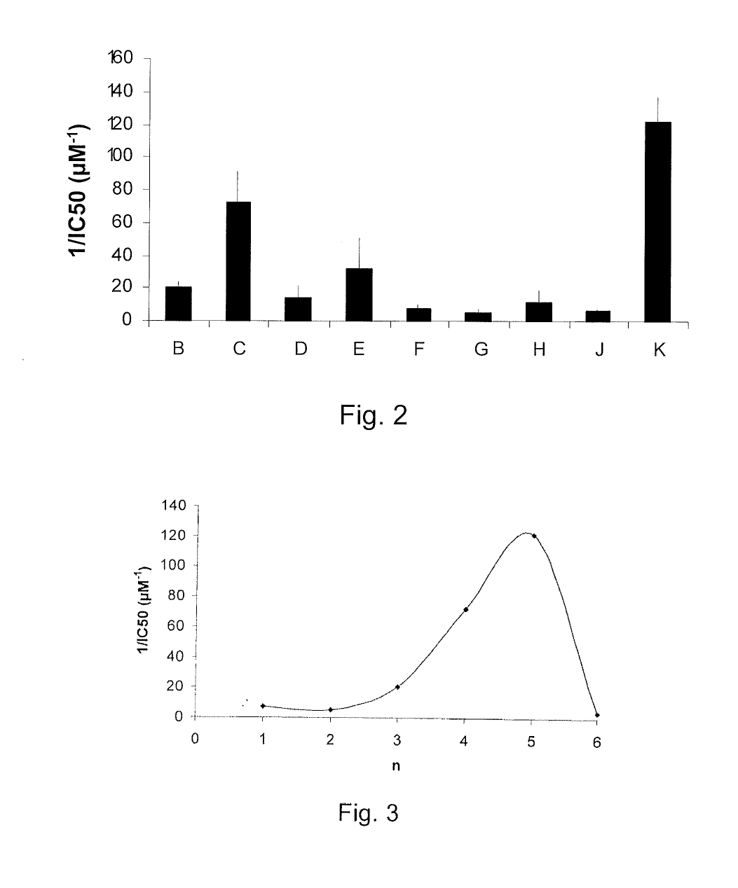

FIG. 2 represents a graph showing the inhibition capability (1/IC₅₀ values) of various compounds of the invention towards the enzyme myeloperoxidase. It can be understood from this graph that the compounds of the present invention show surprinsingly satisfying results. In particular, the compound C (3-(4-Aminobutyl)-5-fluoro-[1H]-indole oxalate) and compound K (3-(5-aminopentyl)-5-fluoro-1H-indole oxalate) showed an unexpected IC₅₀ value of 15±4 nM and 8±1 nM respectively.

FIG. 3 represents a graph showing the inhibition capability (1/IC₅₀ values) of various compounds of the invention towards the enzyme myeloperoxidase. The inhibition capability was evaluated for compounds of formula (Ia) wherein R¹, R² and R were hydrogen and n was 1 to 6. Table 2 reports IC₅₀ and 1/IC₅₀ values in function of lateral chain length for compounds of formula (Ia) wherein R¹, R² are hydrogen and R⁵ is hydrogen in each of the n units.

TABLE 2

| n | IC₅₀ (μM) | 1/IC₅₀ (μM⁻¹) |
|---|---|---|
| 1 | 0.9 ± 0.3 | 7 ± 8 |
| 2 | 0.20 ± 0.03 | 5 ± 0.6 |
| 3 | 0.050 ± 0.008 | 20 ± 4 |
| 4 | 0.015 ± 0.004 | 72 ± 19 |
| 5 | 0.008 ± 0.002 | 122 ± 15 |
| 6 | 0.26 ± 0.01 | 3.8 ± 0.2 |

FIG. 3 and table 2 show that excellent results are achieved when lateral chain length was 3, 4 or 5. Optimum performance was achieved when lateral chain length contains 5 carbon atoms, which corresponds to compound K. Without to be bound by the theory, it is demonstrated that carbon chain elongation dramatically affect the inhibition of the myeloperoxidase, as clearly demonstrated in the assay. Adding alkyl groups on the amino moiety of 5-fluoroindole and/or adding hydrocarbyl group between the indole and the amino moiety, allow to enhance the inhibition capacity of 3-alkyl-5-fluoroindole derivatives towards MPO.

### Low-Density Lipoproteins Oxidation Inhibition

As previously mentioned, the myeloperoxidase oxidizes apoB100 of LDLs (Low density lipoproteins) and apoAl of HDLs (High density lipoproteins) in physiologic conditions. This oxidation couls lead to atherosclerosis. The ability of myeloperoxidase inhibition under physiologic conditions was evaluated in presence of low-density lipoproteins (LDL). The aim was to dramatically limit the oxidation of the low-density lipoproteins under physiologic conditions and, thus, to demonstrate the surprising ability of compounds of the present invention to achieve therapeutic effect in cardiovascular diseases such as atherosclerosis.

### Preparation of the Recombinant Enzyme and Obtaining of LDL.

Recombinant MPO was prepared as previously described. Each batch solution is characterized by its protein concentration (mg/ml), its activity (U/ml), and its specific activity (U/mg). The chlorination activity was determined according to Hewson and Hager. Human plasma served for the isolation of LDL by ultracentrifugation according to Havel et al. Before oxidation, the LDL fraction (1.019<d<1.067 g/ml) was desalted by two consecutive passages through PD10 gel-filtration columns (Amersham Biosciences, The Netherlands) using PBS buffer. The different steps were carried out in the dark and the protein concentration was measured by the Lowry assay for both MPO and LDL.

### Inhibition of LDL Oxidation.

The LDL oxidation was carried out at 37° C. in a final volume of 500 μl. The reaction mixture contained the following reagents at the final concentrations indicated between brackets: pH 7.2, PBS buffer, MPO (1 μg/ml), LDL (1000 μg/ml), 2 μl HCl 1N (4 mM), a compound of formula (Ia) of the present invention (50, 100 and 1000 nM), and H₂O₂ (100 μM). The reaction was stopped after 5 min by cooling the tubes in ice. The assay was performed as described by Moguilevsky et al. (Moguilevsky N., Zouaoui Boudjeltia Z., Babar S., Delrée P., Legssyer I., Carpentier Y., Vanhaeverbeek M., Ducobu J. Monoclonal antibodies against LDL progressively oxidized by myeloperoxidase react with ApoB-100 protein moiety and human atherosclerotic lesions. Biochem. Biophys. Res. Commun. 2004, 323, 1223-1228) in a NUNC maxisorp plate (VWR, Zaventem, Belgium): 200 ng/well of LDL was coated overnight at 4° C. in a sodium bicarbonate pH 9.8 buffer (100 μl). Afterwards, the plate was washed with TBS 80 buffer and then saturated during 1 h at 37° C. with the PBS buffer containing 1% BSA (150 μl/well). After washing the wells twice with the TBS 80 buffer, the monoclonal antibody Mab AG9 (200 ng/well) obtained according to a standard protocol and as previously described was added as a diluted solution in PBS buffer with 0.5% BSA and 0.1% of Polysorbate 20. After incubation for 1 h at 37° C., the plate was washed four times with the TBS 80 buffer and a 3000 times diluted solution of Ig G anti-mouse Alkaline Phosphatase (Promega, Leiden, The Netherlands) in the same buffer was added (100 μl/well). The wells were washed again four times and a revelation solution (150 μl/well) containing 5 mg of para-nitrophenyl phosphate in 5 ml of diethanolamine buffer was added for 30 min at room temperature. The reaction was stopped with 60 μl/well of NaOH 3 N solution. The measurement of the absorbance was performed at 405 nm with a background correction at 655 nm with a Bio-Rad photometer for a 96-well plate (Bio-Rad laboratories, CA, USA). Results were expressed as means of the percentage of LDL oxidation for six independent measurements.

Table 3 reports the percentage of LDL oxidation for different concentrations in compounds of the present invention. Compound A corresponds to 5-fluorotryptamine and is a comparative example. The variability in the method may lead to a percentage of LDL oxidation higher than 100%.

TABLE 3

| | LDL oxidation (%) | | |
|---|---|---|---|
| Compound | 1000 nM | 100 nM | 50 nM |
| A | 41.5 | 111 | 119 |
| B | 9 | 19 | 26 |
| C | 6.16 | 17 | 18.4 |
| D | 9.9 | 64.2 | 91.7 |
| E | 12.1 | 47.9 | 49.2 |
| F | 8 | 60.2 | 80.1 |
| G | 10.2 | 24.8 | 32.9 |
| H | 8.4 | 39.7 | 42.4 |
| J | 5.6 | 42.5 | 79.9 |
| K | 3.2 | 23.4 | 29.5 |
| L | 18.2 | 56.1 | 91.8 |

5-fluorotryptamine was poorly effective or efficient at all to inhibit LDL oxidation irrespective of its concentration in the medium. On the contrary, excellent results were obtained for compounds of the present invention at 1000 nM. In particular, compound K and C allowed to inhibit LDL oxidation in a way that would not be expected. Indeed, only 3.2% and 6.1% of LDL oxidation were obtained with compound K and C respectively. Even at low concentration, compounds K and C showed powerful performance. Less than 18.5% of LDL oxidation was obtained for compound C at 100 nM and 50 nM. In addition at low concentrations, compounds G and B showed excellent results. This experiment highlights that compounds of the present invention are expected to show powerful therapeutic activity in cardiovascular diseases in which myeloperoxidase activity is harmful. Compounds of formula (Ia) according to the present invention, and in particular compounds K and C, have excellent antioxidant properties under physiologic conditions which would not be expected.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. Compounds of formula (Ia) 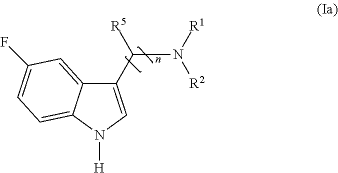
wherein
n is an integer between 2 and 10,
R¹ and R² independently represent a substituent selected from the group consisting of hydrogen, C₁-C₁₀ alkyl, C₃-C₁₀ cycloalkyl and aminoalkyl, or R¹ and
R² are taken together with the nitrogen atom to which they are attached to form a four to ten-membered heterocycle,
R⁵ represents independently in each of the n units a substituent selected from the group consisting of hydrogen, C₁-C₁₀ alkyl, halogen, alkoxy, aminoalkyl and alkylamino,
or pharmaceutically acceptable salts thereof, with the proviso that 5-fluorotryptamine is excluded, for the treatment or the prophylaxis of neuroinflammatory diseases or disorders.

2. Compounds according to claim 1, wherein n is an integer between 2 and 6.

3. Compounds according to claim 1, wherein R⁵ represents independently in each of the n units a substituent selected from the group consisting of hydrogen and C₁-C₆ alkyl ; R¹ and R² independently represent a substituent selected from the group consisting of hydrogen and C₁-C₆ alkyl, or R¹ and R² are taken together with the nitrogen atom to which they are attached to form a heterocycle selected from the group consisting of azetidine, pyrrolidine, piperidine, piperazine, azepane and azocane.

4. Compounds according to claim 1, wherein R⁵ represents independently in each of the n units a substituent selected from the group consisting of hydrogen and C₁-C₂ alkyl ; R¹ and R² independently represent a substituent selected from the group consisting of hydrogen and C₁-C₂ alkyl, or R¹ and R² are taken together with the nitrogen atom to which they are attached to form a heterocycle selected from the group consisting of pyrrolidine and piperazine.

5. Compounds according to of claim 1, wherein said compounds are selected from the group consisting of 3-(3-Aminopropyl)-5-fluoro-[1H]-indole, (3-(4-Aminobutyl)-5-fluoro-[1H]-indole, (5-Fluoro-3-[2-(1-pyrrolidinyl)ethyl]-[1H]-indole, (N,N-Dimethyl-3-(2-aminoethyl)-5-fluoro-[1H]-indole and 3-(5-aminopentyl)-5-fluoro-1H-indole oxalate, or a pharmaceutically acceptable salt thereof.

6. Compounds according to claim 1, wherein said compound has an IC₅₀ value equal or less than 0.2 μM against myeloperoxidase enzyme.

7. Compounds according to claim 1, where the neuroinflammatory diseases or disorders are selected from the group consisting of multiple sclerosis, atherosclerosis, Alzheimer's disease, chronic pulmonar disease, chronic inflammatory syndromes linked to joints and Parkinson's disease.

8. Pharmaceutical composition comprising a therapeutically effective amount of a compound of formula (Ia) according to claim 1, or a pharmaceutically acceptable salt thereof, in admixture with a pharmaceutically acceptable adjuvant, diluent or carrier.

9. A method for the treatment or prophylaxis of neuroinflammatory diseases or disorders comprising administering a pharmaceutical composition according to claim 8.

10. The method according to claim 9 where said neuroinflammatory diseases or disorders are selected from the group consisting of multiple sclerosis, atherosclerosis, Alzheimer's disease, chronic pulmonar disease, chronic inflammatory syndromes linked to joints and Parkinson's disease.

11. Method for inhibiting myeloperoxidase enzyme activity comprising the step of adding a compound according to any one of claim 1 to a medium containing said enzyme, said compound being added in a concentration effective to inhibit the activity of said enzyme.

12. (canceled)

13. Method for the treatment of atherosclerosis comprising the step of administering a therapeutically effective amount of a compound of formula (Ia) according to claim 1 or a therapeutically effective amount of a pharmaceutical composition according to claim 8, to a patient in need thereof.

14. Method for inhibiting low density lipoproteins (LDL) oxidation comprising the step of contacting a compound of formula (Ia) according to claim 1 in a medium containing said low density lipoproteins and myeloperoxidase enzyme.

15. (canceled)
